(19) United States
(12) Patent Application Publication
(10) Pub. No.: US 2014/0356387 A1
(43) Pub. Date: Dec. 4, 2014

(54) METHOD FOR INDUCING IMMUNE TOLERANCE THROUGH TARGETTED GENE EXPRESSION

(71) Applicant: BLOODCENTER RESEARCH FOUNDATION, Milwaukee, WI (US)

(72) Inventors: Qizhen Shi, Brookfield, WI (US); Robert R. Montgomery, Cedarburg, WI (US)

(21) Appl. No.: 14/362,677

(22) PCT Filed: Dec. 5, 2012
(86) PCT No.: PCT/US12/68155

§ 371 Date: Jun. 4, 2014

Related U.S. Application Data
(60) Provisional application No. 61/568,358, filed on Dec. 8, 2011.

(51) Int. Cl. A61K 39/00

(52) U.S. Cl. CPC A61K 39/0008; A61K 2039/5156
USPC 424/184.1

(57) ABSTRACT

A method of inducing immune tolerance against a protein of interest comprising the steps of (a) transducing hematopoietic stem cells with a gene for the protein of interest wherein the gene is operably connected to a platelet specific promoter, and (b) transplanting the transfected cells of step (a) into to a subject, wherein the protein is expressed, and wherein the subject develops immune tolerance against the protein.

### CROSS-REFERENCE TO RELATED APPLICATIONS

This application claims priority from U.S. Provisional Patent Application 61/568,358 filed Dec. 8, 2011, which is incorporated by reference herein.

### STATEMENT REGARDING FEDERALLY SPONSORED RESEARCH OR DEVELOPMENT

N/A

### BACKGROUND OF THE INVENTION

Hemophilia A, also known as factor VIII deficiency, is commonly treated through replacement therapies involving clotting factor concentrates containing factor VIII derived from plasma or recombinant protein(s). The therapeutic administration of replacement clotting factor can be complicated by patient antibody responses to the protein of interest. Since these patients do not recognize the replacement protein as a self-antigen, the development of inhibitory antibodies, called “inhibitors”, can be a major clinical problem rendering protein replacement therapy useless. Various immune tolerizing induction (ITI) approaches have been studied including the use of high doses of the protein of interest and the use of drugs like rituximab (anti-CD20) with some successes. The use of high doses of recombinant factor VIII for a period of 2-3 years has been shown to be effective in 70-85% of patients [14]. High dose therapy remains costly and even though these ITI strategies work for some patients, a large proportion of patients experience refractory complications from inhibitors. Alternate ways to induce immune tolerance in hemophilia patients and others experiencing complications from antibody reactions are needed.

Gene therapy involves the genetic manipulation of genes responsible for disease. One possible approach for patients, like those with hemophilia deficient for a single functional protein, is the transmission of genetic material encoding the protein of interest. Many technical issues remain a problem including control of the gene insertion site, the control of gene expression, and others that can confound expression of the correct protein, at the correct time and in the correct location.

### SUMMARY OF THE INVENTION

In one embodiment, the present invention is a method of inducing immune tolerance against a protein of interest comprising the steps of: (a) transducing hematopoietic stem cells with a gene for the protein of interest wherein the gene is operably connected to a platelet specific promoter, and (b) transplanting the transfected cells of step (a) into a subject, wherein the protein is expressed, and wherein the subject develops immune tolerance against the protein. In some embodiments, the subject is immunologically sensitized to the protein prior to step (a). In some embodiments, the subject is not immunologically sensitized to the protein prior to step (a). In some embodiments, the transplanting of step (b) is a bone marrow transplant or an intravenous infusion of hematopoietic stem cells.

In some embodiments, the platelet specific promoter is the CD41 integrin alphaIIb (αIIb) promoter. In other embodiments, the platelet promoter is selected from the group consisting of glycoprotein VI promoter, platelet factor 4 (PF4) promoter, glycoprotein Ib alpha promoter, glycoprotein Ib beta promoter, glycoprotein IX promoter and other platelet protein promoters.

### DESCRIPTION OF THE FIGURES

FIG. 1 illustrates immune response in FVIII null mice that received 2bF8 transgenic platelet infusion. Platelets that contain FVIII from transgenic mice were infused into FVIII^{null} mice to about 30 to 50% of total platelets upon infusion. Inhibitor titer was evaluated by the Bethesda assay after each round of platelet-FVIII infusion. The levels of platelet-FVIII activity in recipients were quantitated by a chromogenic assay. FIG. 1A: The diagram of the infusion scheme. FIG. 1B: Platelet-FVIII:C was still detectable in the recipients one week after infusion of 2bF8 transgenic platelets. Bars represent mean±SD. FIG. 1C: The inhibitor titers in recipients after infusion. These results demonstrate that the immune response is not induced by infusion of transgenic platelets that contain FVIII.

FIG. 2 illustrates immune response in the co-transplantation recipients. FVIII^{null} mice were co-transplanted with splenocytes from highly immunized FVIII^{null} mice and BM cells from FVIII^{null} or 2bF8 transgenic mice. At 8 weeks after transplantation, all recipients were challenged with rhFVIII weekly by intravenous injection. The inhibitor titers decreased to an undetectable level in 40% of co-transplanted recipients that received 2bF8 transgenic BM cells even after the rhFVIII challenge. Bars represent mean±SD. FIG. 2A: Platelet-FVIII expression in the co-transplantation recipients. FIG. 2B shows the immune response in the co-transplantation recipients. These results indicate that platelet-derived FVIII does not act as an immunogen in the presence of primed spleen cells.

FIG. 3 illustrates immune response in 2bF8 lentivirus (LV)-transduced FVIII^{null} mice with pre-existing anti-FVIII immunity. FVIII^{null} recipients were immunized by weekly injection of rhFVIII at 50 U/kg intravenously for 4 weeks. One week after the last immunization, mice received 1100 cGy irradiation followed by syngeneic transplantation of 2bF8 LV-transduced or untransduced bone marrow cells from pre-immunized FVIII^{null} donors. Platelets and plasma were collected at various time points for assays. Bars represent mean±SD. FIG. 3A: Platelet-FVIII expression. FIG. 3B: Inhibitor titers at various time points. FIG. 3C: The half-life (t½) of inhibitor titers in BMT recipients. These results demonstrate that platelet-derived FVIII introduced by 2bF8 LV-mediated gene transfer does not evoke an immune response in previously immunized recipients.

FIG. 4 illustrates immune tolerance induced in 2bF8 LV-transduced FVIII^{null} mice. We transplanted 2bF8 LV-transduced pre-immunized HSCs into 660 cGy sub-lethally irradiated naive FVIII^{null} mice. After BM reconstitution, recipients were assessed by platelet lysate FVIII:C assay and the tail clip survival test to confirm the success of genetic therapy. Animals were then challenged with rhFVIII by weekly injection of rhFVIII at 50 U/kg intravenously for 4 weeks. Bars represent mean±SD. FIG. 4A: Platelet-FVIII expression. FIG. 4B: Inhibitor titers in BMT recipients. These results demonstrate that platelet-derived FVIII may induce immune tolerance in hemophilia A mice.

FIG. 5 is a comparison of different viral vectors in use for gene therapy and an overview of their advantages and disadvantages. Adeno-associated viruses are able to integrate with low frequency into chromosome 19. Lentiviruses also infect non-dividing cells.

FIG. 6 shows the 2bF9 transgene analysis. FIG. 6A: PCR detection of 2bF9 transgene. DNA was purified from peripheral white blood cells. An anti-sense primer and a sense transgene-specific primer were used to amplify a 0.35 kb fragment from 2bF9 expression cassette. Another set of primers were used to amplify a 0.32 kb fragment from the wild-type FIX gene. The third set of primers was used to amplify a 0.5 kb fragment from disrupted mouse FIX gene to confirm FIX knout out background. The panel shows results from recipients at least 3 weeks after bone marrow transplantation (BMT). FIG. 6B: Real-time PCR determined the average copy number of 2bF9 transgene per cell in BMT recipients. DNA was purified from peripheral white blood cells and 100 ng of DNA was analyzed for the 2bF9 proviral DNA, with normalization to the Apo B. Bars represent mean±SD. These results demonstrate viable engraftment of 2bF9 genetically modified hematopoietic stem cells in recipients.

FIG. 7(A-H) is immunofluorescent confocal microscopy analysis of 2bF9 transgene expression. Platelets were isolated from untransduced FIX^{null} control mice (top row) or FIX^{null} mice that received 2bF9 LV-transduced HSCs (middle and bottom rows) and stained for hFIX (A,D and G) and murine VWF (B,E and H). The two images were merged (C,F and I) showing hFIX expressed in transduced platelets.

FIGS. 8A and 8B is a flow cytometry analysis of platelets in 2bF8 LV-transduced recipients. FIG. 8A: Expression of hFIX in 2bF9 LV-transduced recipients (middle and lower panels), transgenic control (upper left panel), and FIX^{null} control (upper right panel) from representative experiments. The platelet population was gated with anti-mouse CD41/integin aIIb monoclonal antibody and hFIX expression was analyzed using goat anti-hFIX and AlexaFluor 488-labeled donkey anti-goat polyclonal antibodies. FIG. 8B: About 6 to 38% platelets expressing hFIX protein in FIX^{null} mice that received 2bF9 LV-transduced HSCs. The expression levels of hFIX in the group conditioned with 1100 cGy were not significantly different from the 660 cGy group.

FIG. 9(A-D) is the quantitative evaluation of FIX expression in 2bF9-LV-transdcued recipients. The levels of FIX expression were determined by both ELISA-based antigen assay (FIGS. 9A and 9B) and the chromogenic-based functional activity assay (FIGS. 9C and 9D) on platelet lysates. The results demonstrate that FIX expression was sustained in 2bF9 LV-transduced recipients under either lethal irradiation of 1100 cGy or sub-lethal 660 cGy irradiation

FIG. 10(A-C) is an analysis of sequential bone marrow transplantation. FIG. 10A: FIX antigen levels in primary and secondary recipients. FIG. 10B: FIX activity levels in primary and secondary recipients. FIG. 10C: Average copy number of 2bF9 proviral DNA per cell in recipients. The results demonstrate that long-term engraftment was obtained in 2bF9 LV-transduced recipients, indicating that long-term reconstituting hematopoietic stem cells were successfully modified by 2bF9 lentivirus.

FIG. 11 is a phenotypic correction analysis in 2bF9 LV-transduced recipients. Tail clip survival test was used to analyze FIX^{null} coagulation defect. Greater than 90% animals survived tail clipping after 2bF9 gene therapy. In contrast, only 2 out of 10 FIX^{null} mice survived under the same challenge.

FIG. 12(A-C) demonstrates that FIX specific immune tolerance is induced in 2bF9 LV-transduced FIX^{null} mice. FIG. 12A: Bethesda assay determined the Inhibitor titers in 2bF9 LV-transduced recipients. To investigate whether the immune tolerance was induced in 2bF9 LV-transduced recipients, animals were immunized with recombinant human FIX (rhFIX) at 200 IU/ml by intraperitoneal administration in the presence of adjuvant twice and the inhibitor titers were determined by Bethesda assay. FIG. 12B: The total anti-FIX inhibitory antibodies in 2bF9 LV-transduced recipients. Animals were immunized with rhFIX at 200 IU/ml by intraperitoneal administration in the presence of adjuvant twice and the total anti-FIX antibodies were determined by ELISA assay. FIG. 12C: The titers of anti-ovalbumin (OVA) antibodies. To ensure that the immune system was not defective in the 2bF9 LV-transduced recipients, animals were challenged with ovalbumin adsorbed onto alum and the titers of anti-OVA antibodies were determined by ELISA assay. Both the 2bF9 LV-transduced and FIX^{null} control mice developed high-titer of anti-OVA antibodies. The levels of anti-OVA IgG in the 2bF9 transduced recipients were not significantly different from FIX^{null} mice after the OVA immunization. These results demonstrate that the immune tolerance develops in 2bF9 LV-transduced recipients and that the immune tolerance is FIX-specific.

### DESCRIPTION OF THE INVENTION

One objective of the present invention is to provide a method to induce immune tolerance in patients. The patients who may benefit from this type of immune-tolerizing approach include those with allergies, auto-immune disease, transplant recipients, and those who lack certain self-antigens as in clotting factor deficiencies resulting in hemophilia A or B. Our current data suggest that expression of a gene product when under the control of the glycoprotein IIb promoter, or other platelet-specific promoter, leads to protein expression in CD41 positive cells. When expressed in this way, the gene product not only fails to induce antibodies but causes the recipient to acquire immune tolerance to the gene product, even in a recipient previously sensitized to the protein. We predict that this mechanism of inducing immune tolerance is applicable for other non-self or self antigens with immune reactivity.

Conditions which could be treated using this invention include those diseases where a specific protein or set of proteins is missing and where replacement therapy induces the development of inhibitors. The conditions which can be treated by this invention include hemophilia A and hemophilia B. We show examples herein, with supporting data, of immune tolerance to both factor VIII and factor IX in hemophilic animal models of disease. We predict this immune tolerance-inducing strategy would work in patients where antibody to a single or multiple target proteins is already present. Additionally, we predict that diseases where this immune tolerance approach could work include those where providing a replacement protein causes development of inhibitors or diseases of autoimmunity where immune tolerance to certain self antigens is lost. The diseases which could be treated with this model include: Bernard Soulier Syndrome, achondroplasia, lysosomal storage diseases, sickle cell disease, Coeliac disease, Crohn's disease, multiple sclerosis, diabetes mellitus type 1 (IDDM), systemic lupus erythematosus (SLE), Sjögren's syndrome, Churg-Strauss Syndrome, Hashimoto's thyroiditis, Graves' disease, idiopathic thrombocytopenic purpura, rheumatoid arthritis (RA), lupus, allergies, graft versus host disease, or alloimmunization resulting from solid organ transplantation, bone marrow transplantation (BMT), or blood transfusion.

When using the invention in a medical procedure to induce immune tolerance to a target protein of interest, one would typically provide to the patient a sufficient number of hematopoietic stem cells (HSCs) that were transduced with a vector containing the protein of interest and a promoter selected to drive platelet-specific expression. Highly enriched stem cell populations could require smaller doses of cells per kg for effective treatment. In mice, a single HSC can rescue an animal after lethal radiation. However, it is challenging to determine which nucleated cells or which mononuclear cells are true stem cells versus cells already committed to become lineage specific. For that reason, enrichment of HSC's in populations of cells containing lineage committed progenitor and effector cells remains the most viable method. In these cases, a dose of at least 1,000,000 total nucleated cells per kg of body weight would be an effective transplant dose.

To collect HSCs from the patient one could perform a surgical bone marrow aspiration or mobilize the peripheral blood with a cytokine such as granulocyte colony stimulating factor such that HSC's would migrate from the bone marrow into the periphery where they could be harvested by venipuncture. Next, one would purify the resulting cells to enrich the cells for HSCs through positive or negative selection means. One could enrich the mobilized peripheral blood or bone marrow populations by positively selecting cells expressing known stem cell markers such as CD34⁺, C-kit, Thy1.1^{+/lo}, Slamf1/CD150⁺ or others.

The following references speak to current practices in using cd34+ selection:

Kasow K A, Sims-Poston L, Eldridge P, Hale G A. Biol Blood Marrow Transplant. 2007 May; 13(5):608-14; Yannaki E, Papayannopoulou T, Jonlin E, Zervou F, Karponi G, Xagorari A, Becker P, Psatha N, Batsis I, Kaloyannidis P, Tahynopoulou V, Constantinou V, Bouinta A, Kotta K, Athanassiadou A, Anagnostopoulos A, Fassas A, Stamatoyannopoulos G. Mol Ther. 2012 January; 20(1):230-8; Jaing T H, Hung I J, Yang C P, Chen S H, Chung H T, Tsay P K, Wen Y C. Bone Marrow Transplant. 2012 January; 47(1):33-9; and Villa I, Kvale E O, Lund-Johansen F, Olweus J. Cytotherapy. 2007; 9(6):600-10.

One could use magnetic bead negative selection to remove cells committed to a lineage expressing any of a number of lineage specific markers such as CD2, CD3, CD4, CD5, CD8, NK1.1, CD11b, CD11c, CD14, CD16, CD19, CD20, CD24, CD56, CD66b, or B220, TER-119, or glycophorin A and conventional means such as magnetic bead kits. Cell populations enriched for HSCs would next be transduced with the vector containing the genetic material of the protein of interest.

A similar approach to transduce and transplant HSC from alternate sources would also be successful. HSCs harvested from the patient to be treated, a cord blood source, a related donor, or an un-related donor with appropriately matched HLA would be successful with this method.

One would splice together, preferably in a viral or other construct, the platelet specific promoter such as the glycoprotein IIb promoter, glycoprotein Ib alpha promoter, glycoprotein Ib beta promoter, platelet factor 4 (PF4) promoter, glycoprotein VI promoter, glycoprotein IX promoter or other platelet protein promoters and next the target protein gene in reading frame.

By “platelet specific”, we mean expression specifically in the platelet, megakaryocyte and/or megakaryocyte progenitors. See, for example, Lichtman et al. (2006) Williams Hematology 7th ed. (1597-1599) New York, N.Y.; McGraw-Hill Medical Publishing.

One benefit of using the IIb promoter is that it has expression in early hematopoiesis. The other platelet specific promoters may not tolerize as well because their expression is at later stages in hematopoiesis closer to platelet maturity. A chimeric intron (β-globin/IgG) is inserted between the promoter and the transgene. It has been demonstrated that areas of the target protein can be deleted and immune tolerance is still effectively produced. One such example of the deletion of target areas is the B domain deletion in the factor VIII expression cassette. Target proteins could be modified to contain post-translational modifications, or other filler sequences like intronic sequences could be incorporated into the construct to enhance the transgene expression. One example of intronic enhancer augmenting transgene expression is the truncated factor IX intron 1 in factor FIX expression cassette.

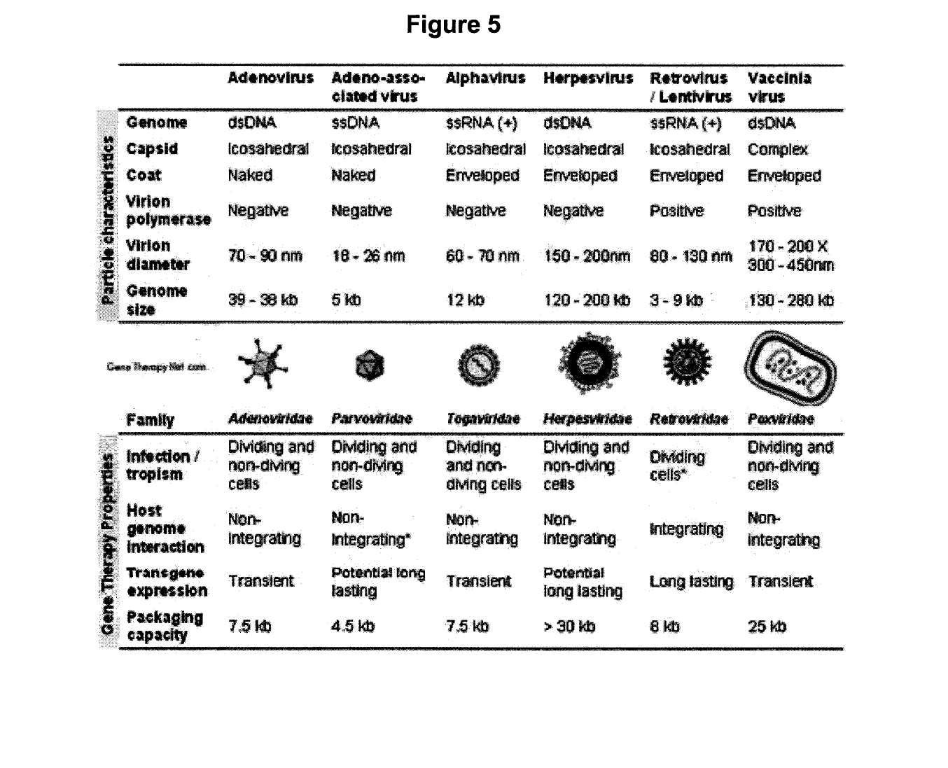

One could use a number of vector or viral vector classes to deliver the DNA coding for the target protein that could include most preferably lentivirus or retrovirus. Additionally other vectors would work, such as adeno-associated virus, adenovirus, herpes simplex virus, liposomes, or naked DNA. Advantages and disadvantages of each class can be found in FIG. 5. Cells could then be tested for the presence of the transgene by conventional PCR.

Following this the HSCs would be transduced with the virus or construct using infection or electroporation. For transduction, HSCs could be pre-stimulated with cytokine cocktail for 24 to 48 hours followed by viral infection in the presence of polybrene and cytokines twice within 48 hours. Infection rates would be between 1 and 1000 viral particles per cell, most preferably 1 to 100 viral particles per cell and most preferably 1-20 viral particles per cell.

Next one would perform a bone marrow or HSC transplant on patients using conventional methods known to those of skill in medicine. Briefly, one could pre-condition the patient using most preferably a sub-lethal dose of total body irradiation or chemotherapy such as busulfan supplemented with anti-thymocyte globulin. Next one would give to the patient by intravenous infusion the prepared HSCs containing the transgene of the target protein.

After transplantation and bone marrow reconstitution, one would use conventional PCR and quantitative real-time PCR to determine the viability of genetically modified engraftment and the copy number of proviral DNA per cells. Next one would use assays such as immunofluorescence confocal microscopy, antigen assay, activity assay, and/or flow cytometry, to determine the transgene protein expression in platelets. One would expect that greater than 5% cells would be genetically modified after transduction and transplantation, resulting in greater than 5% platelets expressing transgene protein. This should be sufficient to induce immune tolerance. The transduction efficiency and expression levels might vary depending on the size of protein that is targeted and the disease model.

The induction of immune tolerance in a patient would be characterized first by viable engraftment of the transduced stem cells. One could use as a readout of engraftment either mismatched HLA markers in the case of non-autologous grafts, the protein of interest, or other molecular markers cloned into the vector. Second, one would be able to detect the protein of interest in circulating platelets as a measure of successful transplant. In the case of factor replacement therapy, functional readouts, like bleeding correction would be a useful readout. Other proteins may have additional readouts available. Third, the lack of antibody development to the target protein, or a reduction in antibody level from prior to the transplant, would be indicative a successful immune tolerization. Last, the lack of antibody production in the patient even after challenge with the protein of interest would be an indicator of longer term induction of immune tolerance. Standard ELISA procedures would be used to detect patient antibody formation. In the case where immune tolerance induction was unsuccessful, one would see antibody formation or a rise in antibody titer to the protein of interest, clearance of the protein of interest, lack of function of the protein of interest or complement mediated effects of clearance.

In a preferred embodiment, the present invention is a method of inducing immune tolerance to a protein of interest through the use of a gene therapy approach targeting expression of the protein of interest or set of proteins of interest inside cells of the megakaryocyte lineage, including platelets.

In another embodiment, the invention is a method of inducing immune tolerance through use of the glycoprotein IIb promoter to target expression of any protein or proteins of interest in cells such as CD41-positive cells of the megakaryocyte lineage, including promegakaryocytes, megakaryocytes, and platelets.

In another embodiment, the invention is a method of inducing immune tolerance in cells of the megakaryocyte lineage, including promegakaryocytes, megakaryocytes, and platelets.

In another embodiment, the invention is a method of inducing immune tolerance to a clotting factor protein whereby genetically modified stem cells express the clotting factor of interest and this clotting factor is expressed under a specific promoter active in hematopoietic cells.

In another embodiment, the invention is a method of inducing immune tolerance using the glycoprotein IIb promoter to target expression of the factor VIII protein, the factor IX protein, glycoprotein Ib, or a fragment, or chimeric protein thereof in cells of the megakaryocyte lineage.

In another embodiment, the invention is a method of inducing immune tolerance using promoters specific to cells of the megakaryocyte lineage.

In another embodiment, the invention is a method of inducing immune tolerance using a promoter and cell type which naturally expresses a chaperone protein to the protein of interest.

By “hematopoietic stem cell (HSC),” we mean any cell that has the functional ability to repopulate the hematopoietic system and self-renew. There are three main sources of HSC including the bone marrow (BM), peripheral blood (PB), and cord blood (CB). A variety of methods exist to harvest and purify HSC from a patient. At the time of this writing, there remains considerable debate as to the true nature of HSC and their surface markers. Those transplanting patients can take approaches that infuse a population of cells which contain HSC and more differentiated hematopoietic cells. Alternatively, one can employ a purification or enrichment strategy based on CD marker selection of HSC. Selection methods commonly use antibodies or can use other binding partner proteins which bind the CD marker of interest. Cells are further purified through the use of linking the antibody or binding partner to magnetic beads, columns, or other solid surface means of capturing cells of interest.

One method employed for HSC purification is the use of CD34+ selection. Another method employed for HSC purification is the use of CD49f positive selection. Another method employed for HSC purification employed for HSC purification is the use of Lin negative selection. Another method employed for HSC purification is the use of CD90 positive selection. Another method employed for HSC purification is the use of CD45 RA negative selection. Another method used is the use of CD38 negative selection. Another method employed for HSC purification is a combination of any of the above selection criteria.

### EXAMPLES

### Factor VIII Example

Our previous studies have shown that targeting FVIII expression to platelets (2bF8) can correct the hemophilia A phenotype in mice even in the presence of inhibitory antibodies. In the present study, we wanted to examine 1) whether platelets containing FVIII can act as an immunogen; and 2) whether platelet-derived FVIII can induce immune tolerance in a hemophilia A mouse model.

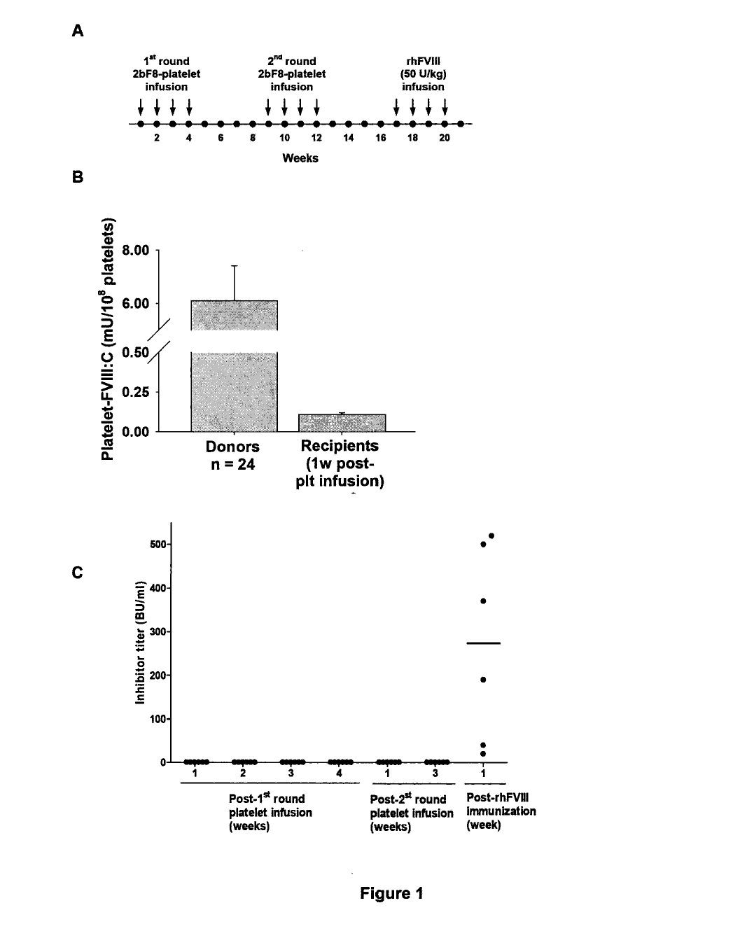

To investigate whether platelets containing FVIII can act as an immunogen in hemophilia A mice, we infused platelets that contains FVIII from transgenic mice with a level of platelet-FVIII of 6 milli unit (mU) per 10⁸ platelets to naive FVIII^{null} mice weekly for 8 weeks (FIG. 1). These platelets were between 30 to 50% of total platelets upon infusion and the levels of platelet-FVIII in the infused animals were 0.11±0.01 mU/10⁸ platelets (n=6) one week after infusion. No anti-FVIII inhibitory antibodies were detected in the infused mice during the course of the study, indicating that infusion of platelets containing FVIII does not trigger immune response in hemophilia A mice. However, all animals developed inhibitors following further challenge with recombinant human FVIII (rhFVIII) at a dose of 50 U/kg by intravenous injection weekly for 4 weeks.

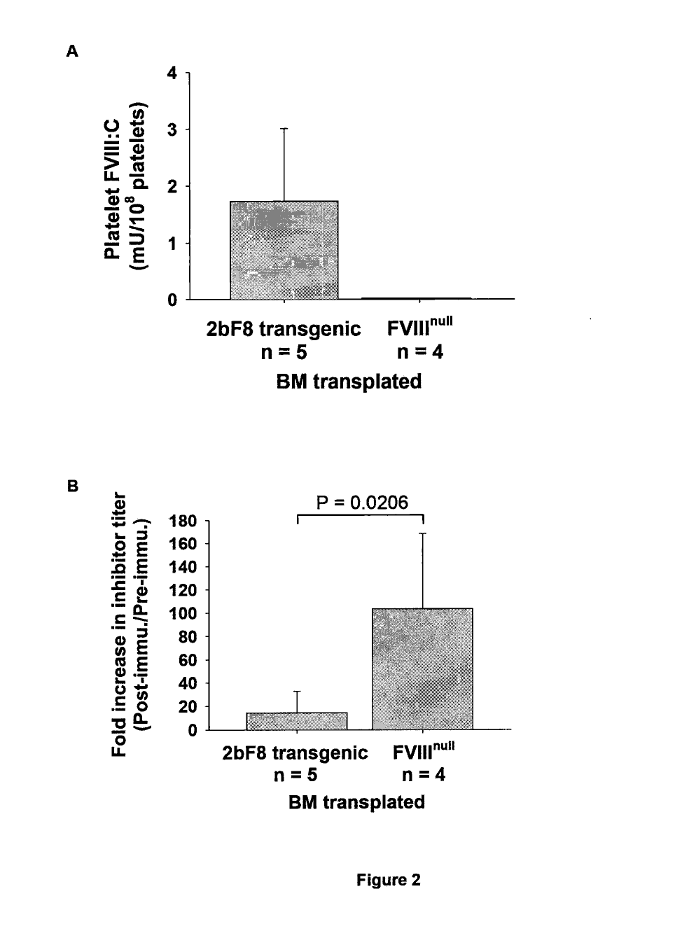

To examine whether platelet-derived FVIII will act as an immunogen in the presence of primed spleen cells from mice already producing inhibitory antibodies, we transplanted splenocytes from highly immunized FVIII^{null} mice and bone marrow (BM) cells from 2bF8 transgenic mice into 400 centi Gray (cGy) sub-lethal irradiated FVIII^{null} recipients (FIG. 2). We monitored the levels of inhibitory antibodies in recipients for up to 8 weeks and found that inhibitor titers declined with time after transplantation. We then challenged co-transplantation recipients with rhFVIII and found that inhibitor titers in the control group co-transplanted of FVIII^{null} BM cells increased 103.55±64.83 fold (n=4), which was significantly more than the group receiving 2bF8 transgenic BM cells (14.34±18.48, n=5) (P<0.05). The inhibitor titers decreased to undetectable in 40% of 2bF8 transgenic BM cells co-transplantation recipients even after rhFVIII challenge, indicating immune tolerance was induced in these recipients.

These data indicate that a gene therapy strategy is a viable option to generate ITI in patients experiencing complications from diseases or conditions with an immune mediated component like autoimmunity, transplantation, allergy, and the replacement of certain antigens in naive individuals such as factor VIII therapy for hemophilia A or factor IX therapy for hemophilia B.

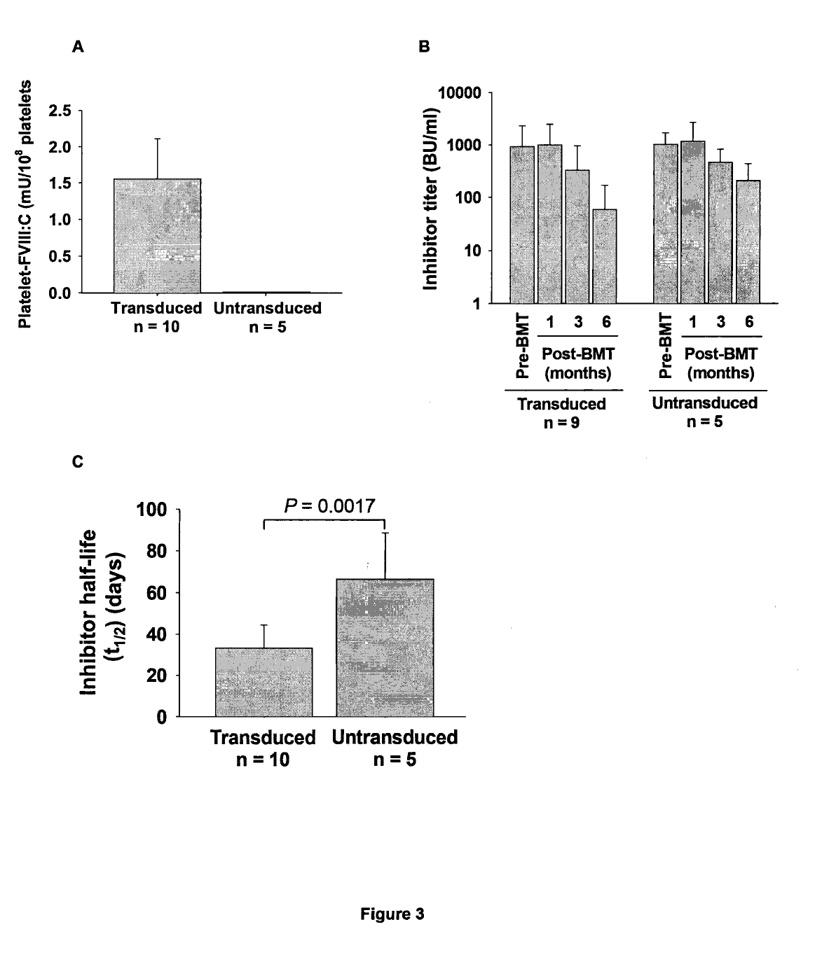

To further explore the immune response in the lentivirus-mediated platelet-derived FVIII gene therapy of hemophilia A mice, we transduced hematopoietic stem cells from pre-immunized FVIII^{null} mice with 2bF8 lentivirus (LV) followed by syngeneic transplantation into pre-immunized lethally irradiated FVIII^{null} recipients and monitored the levels of inhibitor titers in recipients (FIG. 3). Mice were pretreated with FVIII to induce inhibitor formation that would be seen in a patient who would be a candidate for this type of therapy. After BM reconstitution, platelet-FVIII expression was sustained (1.56±0.56 mU/10⁸ platelets, n=10), but inhibitor titers declined with time, indicating that platelet-derived FVIII does not provoke an immune response in FVIII^{null} mice that had previously mounted an immune response to rhFVIII. The t_{1/2} of inhibitor disappearance in 2bF8 LV-transduced recipients (33.65±11.12 days, n=10) was significantly shorter than in untransduced controls (66.43±22.24 days, n=4) (P<0.01).

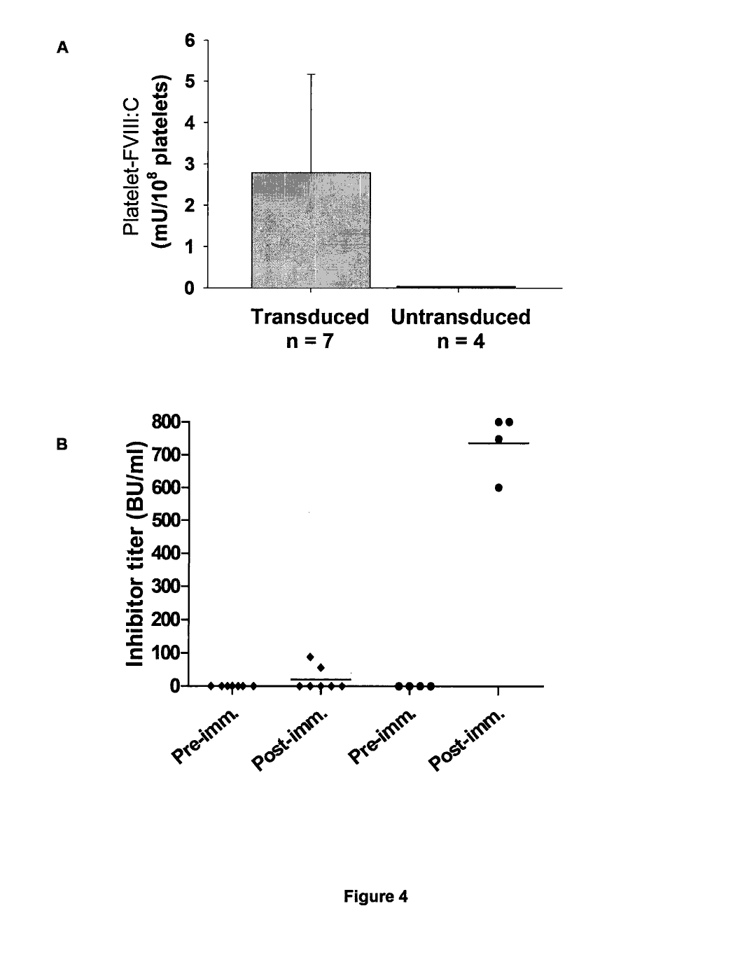

We also transplanted 2bF8 LV-transduced pre-immunized HSCs into 660 cGy sub-lethally irradiated naive FVIII^{null} mice. After BM reconstitution, recipients were assessed by platelet lysate FVIII activity assay and tail clip survival test to confirm the success of genetic therapy. Animals were then challenged with rhFVIII. Only 2 of 7 2bF8 LV-transduced recipients developed inhibitory antibodies at 55 and 87 Bethesda Units/milliliter (BU/ml), while all 4 non-transduced controls developed high titer of inhibitors at 735.50±94.65 BU/ml (FIG. 4). These data indicate that immune tolerance can be induce in hemophilia A patients using a gene therapy approach by expressing FVIII under control of the platelet-specific glycoprotein IIb promoter.

In conclusion, our results from studies with factor VIII demonstrate that 1) platelets containing FVIII are not immunogenic in hemophilia A mice; and 2) platelet-derived FVIII gene therapy induces immune tolerance in hemophilia A mice with or without pre-existing inhibitory antibodies. It would add to the appeal of any genetic therapeutic approach were it to not only improve hemostasis, but also induce immune tolerance toward the replacement protein, particularly in the case of patients with pre-existing immunity. This tolerance induction would add an additional significant benefit to patients with platelet-derived FVIII gene therapy strategy because protein infusion could be administered in some special situations (e.g. surgery in which a greater levels of FVIII may be required) with minimized risk of inhibitor development.

### Factor IX Example

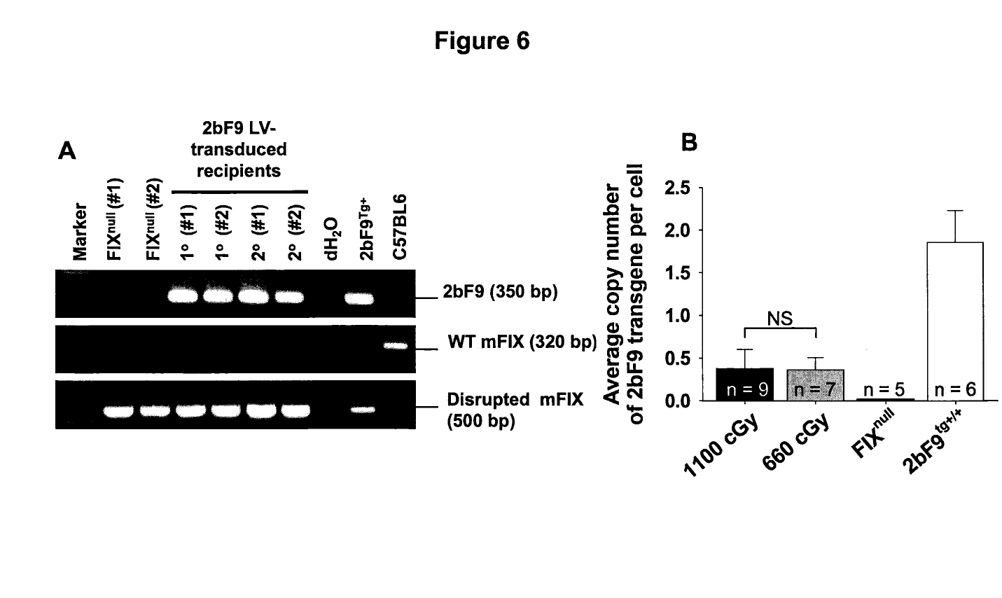

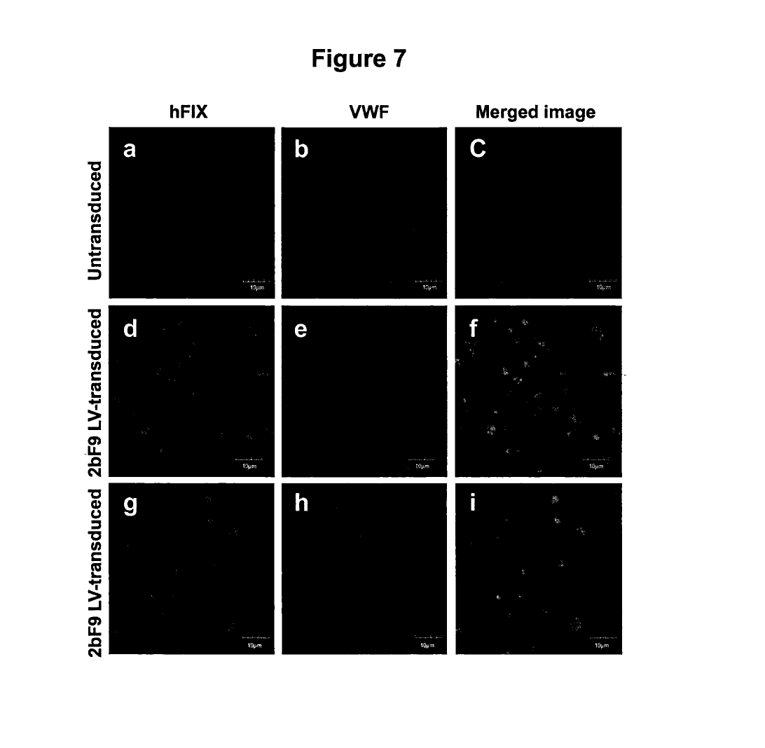

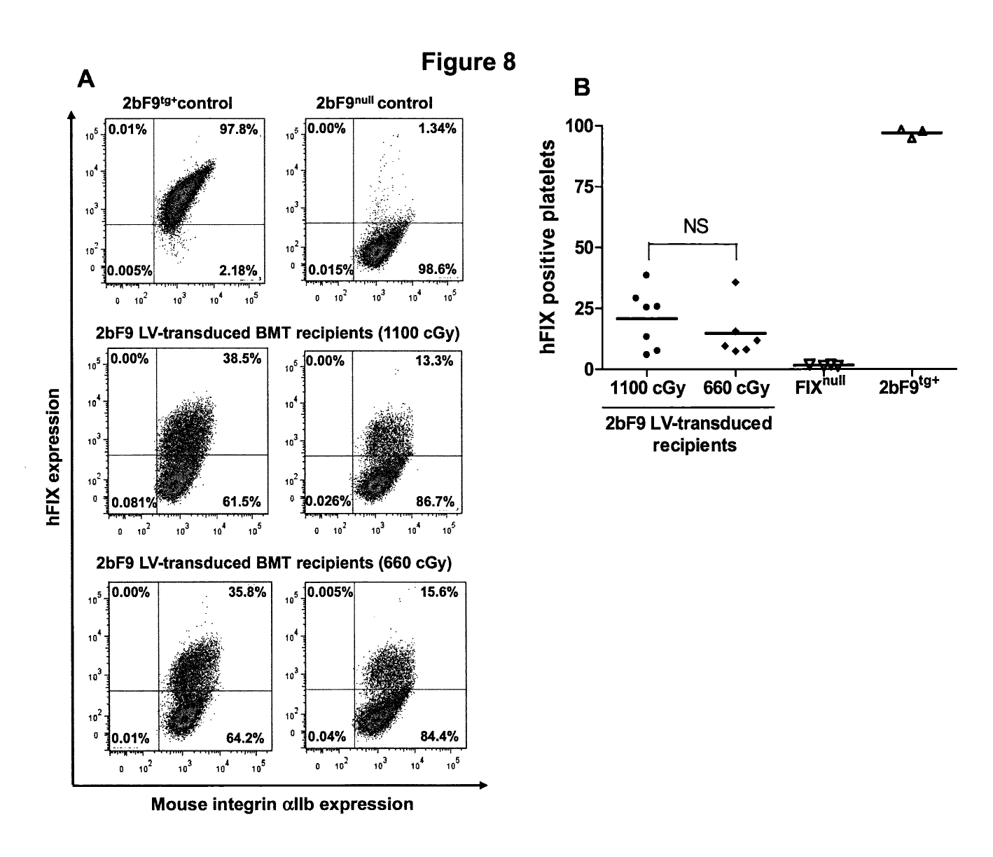

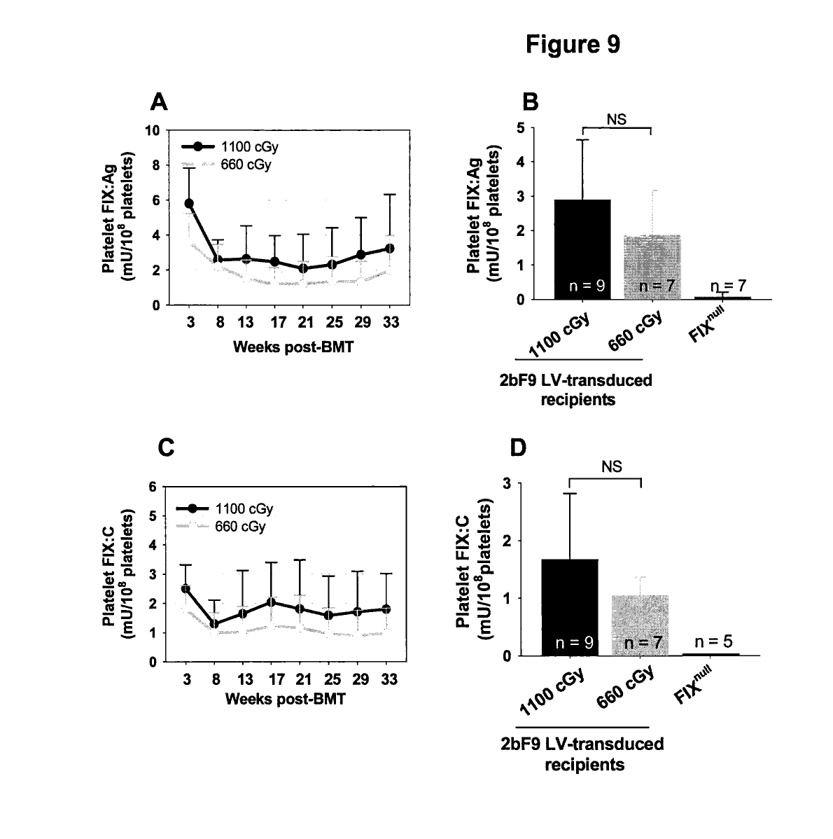

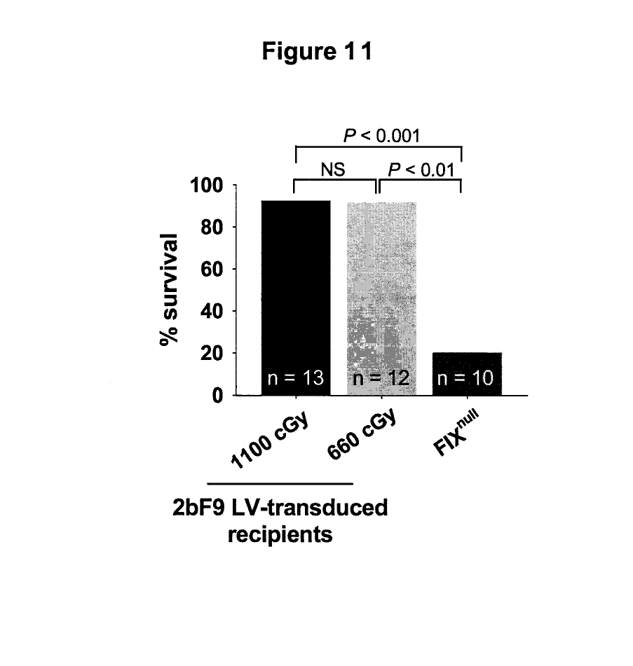

Immune tolerization to factor IX was also demonstrated as effective using a similar approach. While data from the clinical trials using AAV vector expression FIX in hemophilia B gene therapy in humans are very encouraging, for individuals with severe liver disease or neutralizing antibodies to AAV, an alternative gene therapy approach might be desired. Our previous studies have demonstrated that lentivirus-mediated platelet gene therapy can correct murine hemophilia A phenotype, but this approach has not been explored for hemophilia B. In the current study, we developed a clinical translatable approach for platelet gene therapy of hemophilia B. Platelet-FIX (2bF9) expression in hemophilia B (FIX^{null}) mice was introduced by transplantation of hematopoietic stem cells (HSCs) transduced with 2bF9 lentivirus (LV). The recipients were analyzed beginning at 3 weeks after bone marrow (BM) transplantation. Expression of the 2bF9 product was detected by PCR in all recipients that received 2bF9 LV-transduced BM cells, indicating viable engraftment of BM genetically modified with the 2bF9 LV transfer vector (FIG. 6). The expression of the hFIX transgene protein in the transduced platelets was confirmed by immunofluorescent confocal microscopy (FIG. 7). Flow cytometry showed that there were 20.8±12.1% (n=7) and 14.8±10.7% (n=6) 2bF9 LV-transduced platelets respectively in the recipients preconditioned with 1100 cGy or 660 cGy (FIG. 8). The antigen levels of FIX (FIX:Ag) were 2.89±1.75 mU/10⁸ platelets (n=9) in the recipients preconditioned with 1100 cGy and 1.87±1.30 mU/10⁸ platelets (n=7) in the 660 cGy group, while the activity (FIX:C) levels were 1.67±1.15 and 1.13±0.85 mU/10⁸ platelets respectively (FIG. 9). There was a small amount of FIX detected in the 2bF9 LV-transduced recipient plasma with the average levels of 2.22 mU/ml in 1100 cGy group and 1.44 mU/ml in 660 cGy group. To analyze the distribution of the FIX between platelets and plasma, we normalized FIX levels to total whole blood FIX content. The results demonstrated that 90% to 95% of whole blood FIX was stored in platelets. The tail clip survival test demonstrated that 15 out of 16 mice that received 2bF9 LV-transduced HSCs survived the tail clip challenge, while 8 out of 10 FIX^{null} control mice died after tail clipping (FIG. 11).

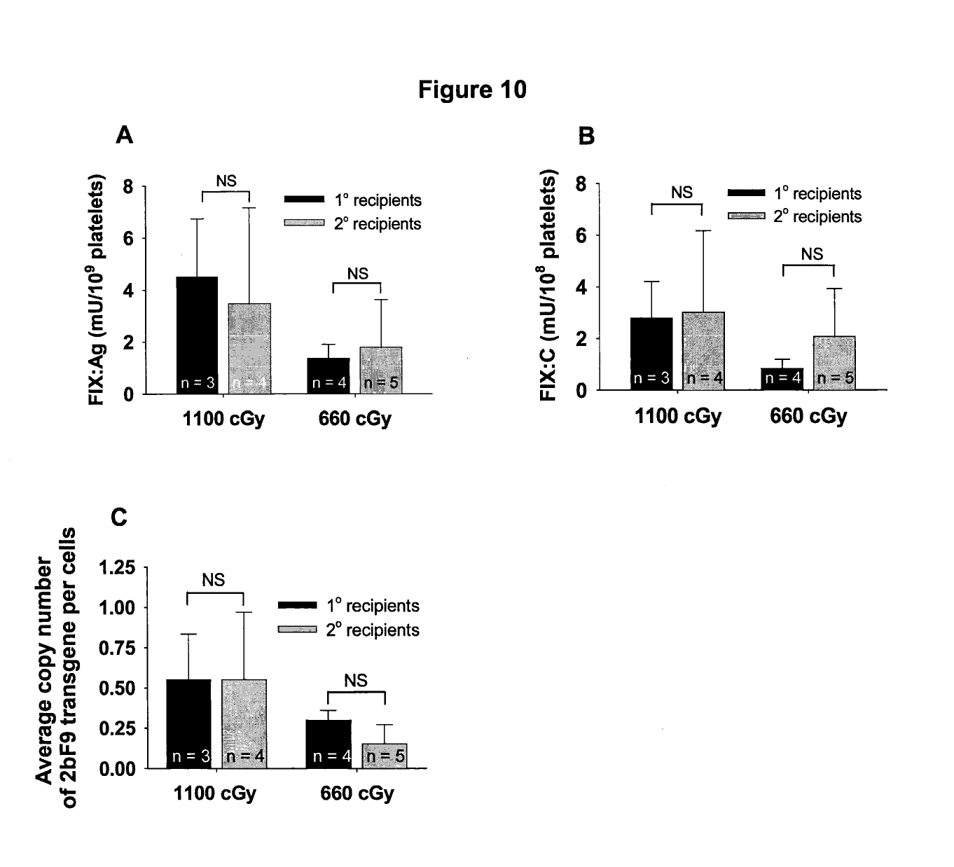

Nine months after transplantation, sequential transplantation was performed on some of the primary recipients (FIG. 10). Platelet-hFIX expression in the secondary recipients was sustained, leading to phenotypic correction and confirming that long-term engrafting HSCs were successfully transduced with 2bF9 LV. Notably, none of the transduced recipients developed anti-FIX antibodies after platelet gene therapy.

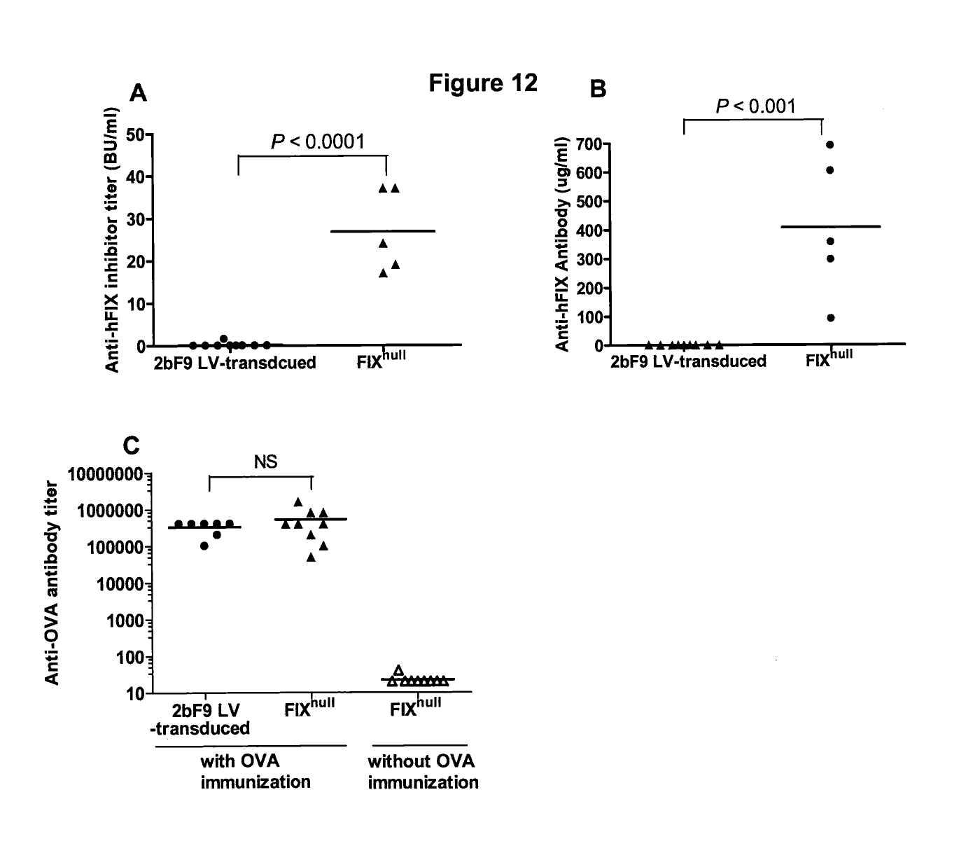

To investigate whether immune tolerance was induced in 2bF9 LV-transduced recipients, we challenged the recipients with recombinant human FIX (rhFIX) in the presence of adjuvant. Only 1 out of 9 2bF9 LV-transduced recipients developed a low titer of inhibitory antibodies (1.6 BU/ml) as measured by a modified Bethesda assay. In contrast, all of the FIX^{null} controls developed inhibitory antibodies ranging from 17-37 BU/ml after the same challenge (n=5) (FIG. 12).

To ensure that the immune system was not defective in the 2bF9 LV-transduced recipients and that the tolerance induction is FIX antigen-specific, we further challenged the animals with ovalbumin (OVA) absorbed on Alum. Both the 2bF9 LV-transduced and FIX^{null} control mice developed high-titer of anti-OVA antibodies. The levels of anti-OVA IgG in the 2bF9 transduced recipients were not significantly different from FIX^{null} mice after the OVA immunization, confirming that tolerance induction in 2bF9 LV-transduced mice is FIX-specific (FIG. 12).

Taken together, our data suggest that lentivirus-mediated bone marrow transduction and transplantation can not only provide sustained phenotypic correction, but also induce immune tolerance in hemophilia B mice, indicating that this approach may be a promising strategy for gene therapy of hemophilia B in humans.

### DISCUSSION

Our previous studies have demonstrated that targeting FVIII expression to platelets (2bF8) corrects the murine hemophilia A phenotype even in the presence of inhibitors. Our further studies have shown that 2bF8 LV-transduced hemophilia A mice develop neither inhibitory nor non-inhibitory antibodies. In the current study, we investigated 1) whether platelets containing FVIII would act as an immunogen; and 2) whether platelet-derived FVIII would induce immune tolerance in hemophilia A mice with or without pre-existing immunity.

Platelet infusion: Naive FVIII^{null} mice were intravenously infused with the platelets that contains FVIII from transgenic mice weekly for total 8 weeks.

Co-transplantation: FVIII^{null} mice conditioned with 400 cGy total body irradiation were co-transplanted with splenocytes from highly immunized FVIII^{null} mice and BM cells from 2bF8 transgenic mice.

2bF8 LV-mediated BM transduction and syngeneic transplantation: 2bF8 LV-transduced pre-immunized HSCs were transplanted into FVIII^{null} mice with or without pre-existing immunity.

The levels of platelet FVIII activity (FVIII:C) were quantitated by a chromogenic assay. Inhibitor titers were determined by the Bethesda assay.

### REFERENCES

1. Shi, Q., Wilcox, D. A., Fahs, S. A., Kroner, P. A., and Montgomery, R. R. Expression of human factor VIII under control of the αIIb promoter in megakaryocytic cell line as well as storage together with VWF. Mol. Genet. And Metab. 2003, 79 (1): 25-33.

2. Shi, Q., Wilcox, D. A., Fahs, S. A., Weiler, H., Well, C. C., Cooley, B. C., Desai, D., Morateck, P. A., Gorski, J., and Montgomery, R. R. Factor VIII ectopically targeted to platelets is therapeutic in hemophilia A with high-titer inhibitory antibodies. J Clin Invest 2006, 116 (7):1974-1982.

3. Shi, Q., Wilcox, D. A., Fahs, S. A., Fang, J., Johnson B. D., Du, L., Desai, D., and Montgomery, R. R. Lentivirus-mediated platelet-derived factor VIII (FVIII) gene therapy of murine hemophilia A. J Thromb Haemost 2007, 5 (2):352-361.

4. Shi, Q., Fahs, S. A., Wilcox, D. A., Kuether, E. L., Morateck, P. A., Mareno, N., Weiler, H., Montgomery, R. R. Syngeneic transplantation of hematopoietic stem cells (HSC) that are genetically modified to express factor VIII (FVIII) in platelets restores hemostasis to hemophilia A mice with pre-existing FVIII immunity. Blood 2008,112 (7):2713-2721.

5. Shi, Q., Fahs, S. A., Kuether, E. L., Cooley, B. C., Weiler, H., Montgomery, R. R. Targeting FVIII expression to endothelial cells regenerates a releasable pool of FVIII and restores hemostasis in a mouse model of hemophilia A. Blood 2010, 116(16): 3049-57.

6. Shi, Q., Montgomery, R. R. Platelets as delivery systems for disease treatments. Adv Drug Deliv Rev. 2010 Sep. 30; 62(12): 1196-203.

7. Montgomery, R, R. and Shi, Q. Alternative Strategies for Gene Therapy of Hemophilia. Hematology Am Soc Hematol Educ Program. 2010; 2010: 197-202.

8. Kanaji, S., Kuether, E. L., Schroeder, J. A., Fahs, S. A., Ware J., Montgomery, R. R., Shi, Q. Lentivirus-mediated gene therapy of Bernard-Soulier Syndrome in a GPIbα□ deficient mouse model. Mol Ther. 2012 March; 20(3):625-32.

9. Kuether, E. L., Cooley, B. C., Fahs, S. A., Schroeder, J. A., Chen, Y., Montgomery, R. R., Wilcox, D. A., Shi, Q. Lentivirus-mediated platelet gene therapy of murine hemophilia A with pre-existing anti-FVIII immunity. J Thromb Haemost. 2012 August; 10(8): 1570-80.

10. Evans, G. L., and Morgan, R. A. Genetic induction of immune tolerance to human clotting factor VIII in a mouse model for hemophilia A. Proc. Natl. Acad. Sci. USA 1998, 95(10): 5734-9.

11. Bagley, J., Bracy, J. L., Tian, C., Kang, E. S., Iacomini, J. Establishing immunological tolerance through the induction of molecular chimerism. Front Biosci. 2002;7:d1331-7.

12. Moayeri M., Hawley, T. S., Hawley, R. G. Correction of murine hemophilia A by hematopoietic stem cell gene therapy. Mol Ther. 2005; 12(6):1034-42.

13. Chang, A. H., Stephan, M. T. and Sadelain, M. Stem cell-derived erythroid cells mediate long-term systemic protein delivery. Nat. Biotechnol 2006, 24: 1017-1021.

14. Valentino L A, Recht M, Dipaola J, Shapiro A D, Pipe S W, Ewing N, Urgo J, Bullock T, Simmons M, Deguzman C. Experience with a third generation recombinant factor VIII concentrate (Advate) for immune tolerance induction in patients with haemophilia A. Haemophilia. 2009 May; 15(3):718-26. Epub 2009 Feb. 27.

15. Weissman, I. L., and Shizuru, J. A. The origins of the identification and isolation of hematopoietic stem cells, and their capability to induce donor-specific transplantation tolerance and treat autoimmune diseases. Blood 2008: 112: 3543-3553).

16. Faiyaz N. et al. Isolation of single human hematopoietic stem cells capable of long-term multilineage engraftment. Science 2011: 333: 218-221.

17. Montgomery, R. R., Shi, Q. Platelet and Endothelial Expression of Clotting Factors for the Treatment of Hemophilia. Thromb Res. 2012 May; 129 Suppl 2:S46-8.

18. Shi, Q., Kuether, E. L., Schroeder, J. A., Perry, C. L., Fahs, S. A., Gil, J. C., Montgomery, R. R. FVIII inhibitors: VWF makes a difference in vitro and in vivo. J Thromb Haemost. 2012; 10(11): 2328-37.

Abstracts:

19. Shi, Q., Wilcox D. A., Fahs S. A., Fang J., Johnson B. D., Weiler H., and Montgomery R. R. Lentivirus-mediated platelet-specific gene therapy of hemophilia A. Blood 2004; 104: 2974.

20. Shi, Q., Wilcox, D. A., Fahs, S. A., Weiler, H., and Montgomery, R. R. Plateletderived factor VIII (FVIII) is protected from inhibitor inactivation—A potential approach for gene therapy of hemophilia A with inhibitors. J Thromb and Haemost 2005; 3, Supplement 1: H05. Accepted for oral presentation at ISTH meeting.

21. Shi, Q., Wilcox, D. A., Fahs, S. A., Fang, J., Johnson, B. D., Weiler, H., and Montgomery, R. R. Murine hemophilia A is phenotypically corrected by plateletexpressed factor VIII even in the absence of detectable plasma FVIII. J Thromb and Haemost 2005; 3, Supplement 1: OR186. Accepted for oral presentation in Hot Topics Talk Session at ISTH meeting.

22. Shi, Q., Wilcox, D. A., Fahs, S. A., Cooley, B. C., Desai, D., Weiler, H., Morateck, P. A., and Montgomery, R. R. Platelet-derived factor VIII (FVIII) corrects the murine hemophilia A phenotype even in the presence of FVIII inhibitors. Blood 2005; 106: 457. Accepted for oral presentation at ASH meeting.

23. Shi, Q., Wilcox, D. A., Fahs, S. A., and Montgomery, R. R. Ectopic expression of FVIII in platelets as a model approach to gene therapy of hemophilia A patients with inhibitors. Pediatrics Academic Society Annual Meeting, San Francisco, 2006. Accepted for oral presentation at PNAS meeting.

24. Shi, Q., Fahs, S. A, Wilcox, D. A., Weiler, H., and Montgomery, R. R. Transplant bone marrow that is genetically modified to express FVIII only in platelets can restore hemostasis to hemophilia A mice on a strong inhibitor background. J Thromb Haemost 2007; 5 supplement 2: P-W-232

25. Shi, Q., Fahs, S. A., Wilcox, D. A., Kuether, E. L., Weiler, H., and Montgomery R. R. In the presence of pre-existing factor VIII (FVIII) immunity, hematopoietic stem cells (HSC) that are genetically modified to express FVIII in platelets were successfully transplanted into hemophilic mice under myeloablative and various non-myeloablative conditions. Blood 2007; 110: 235a. Accepted for oral presentation at ASH meeting.

26. Shi, Q. Platelet and endothelial FVIII/VWF expression in hemophilia gene therapy. The 9th Workshop on Novel Technologies and Gene transfer for Hemophilia, The Children's Hospital of Philadelphia. February, 2008. Invited for a lecture.

27. Shi, Q. Platelet-specific Gene therapy of hemophilia A and hemophilia A with inhibitors. The Physician/Researcher track of NHF's 60th Annual Meeting, Denver, Colo., November 2008. Invited for a lecture.

28. Shi, Q., Kuether, E. L., Cooley, B. C., Fahs, S. A., Schroeder, J. A., Wilcox, D. A., and Montgomery, R. R. Sustained Phenotypic Correction of Murine Hemophilia A with Pre-Existing Anti-FVIII Immunity Using Lentivirus-Mediated Platelet-Specific FVIII Gene Transfer. Blood 2009; 114: 18. Accepted for oral presentation at ASH meeting.

29. Du, L. M., Nichols, T. C., Haberichter, S. L., Jacobi, P. M., Jensen, E. S., Fang, F., Shi, Q., Montgomery, R. R. and Wilcox, D. A. Platelet-Targeted Expression of Human BDD-FVIII within a Canine Model for Hemophilia A Shows Efficacy for Human Clinical Trials. Blood 2009; 114: 289. Accepted for oral presentation at ASH meeting.

30. Shi, Q., Kuether, E. L., Schroeder, J. A., Fahs, S. A., Wilcox, D. A., Montgomery, R. R. The Important Role of Von Willebrand Factor In Platelet-Derived FVIII Gene Therapy of Murine Hemophilia A In the Presence of Inhibitors. Blood 2010; 116: 907. Accepted for oral presentation at ASH meeting.

31. Shi, Q. Platelets as delivery system for gene therapy of hemophilia A and B. BIT Life Sciences' 2nd World DNA and Genome Day, Dalian, China. Apr. 25-29, 2011. Invited for a lecture.

32. Shi, Q. Targeting factor VIII (FVIII) expression to platelets for gene therapy of hemophilia A with inhibitors. The 14th Annual Meeting of American Society of Gene and Cell Therapy, Seattle, May, 2011. Invited for a lecture in the Outstanding New Investigator Symposium.

33. Shi, Q., Kuether, E. L., Schroeder, J. A., Fahs, S. A., Montgomery, R. R. Targeting FVIII Expression to human platelets corrects the hemophilic phenotype in an immunocompromised hemophilia A mouse model transplanted with genetically manipulated human cord blood stem cells. Blood 2011; 118: 20. Accepted for oral presentation at ASH meeting. 34. Chen Y., Kuether, E. L., Schroeder, J. A., Montgomery, R. R., Scott, D. W., and Shi, Q. Targeting FVIII expression to platelets induces immune tolerance in hemophilia A mice with or without pre-existing anti-FVIII immunity. Blood 2011; 118: 4170.

35. Shi, Q. Lentivirus transduction of megakaryocytes: immune protection and human cell studies. 11^{th} NHF New Technologies and Gene Therapy Workshop. The Children's Hospital of Philadelphia, Philadelphia, Pa. Mar. 2-3, 2012. Invited for a lecture.

36. Shi, Q. Kuether, E. L., Schroeder, J. A., Fahs, S. A., Montgomery, R. R. Platelet Gene Therapy Corrects the Hemophilic Phenotype in Immunocompromised Hemophilia A Mice Transplanted with Genetically Manipulated Human Cord Blood Stem Cells. The 58^{th} Annual Meeting of the Scientific and Standardization Committee of the ISTH. Liverpool, United Kingdom. Jun. 27-30, 2012. Accepted for Oral Presentation in the Hot Topic Talk.

37. Shi, Q., Kuether, E. L., Schroeder, J. A., Perry, C. L., Fahs, S. A., Montgomery, R. R. VWF Exerts A Protective Effect on FVIII from Inhibitor Inactivation Both In Vitro and In Vivo. The 58^{th} Annual Meeting of the Scientific and Standardization Committee of the ISTH. Liverpool, United Kingdom. Jun. 27-30, 2012. Accepted for Poster Presentation.

38. Chen Y., Kuether, E. L., Schroeder, J. A., Zhang, G., Montgomery, R. R., and Shi, Q. Lentivirus-mediated Platelet Gene Therapy Corrects Bleeding Diathesis and Induces Immune Tolerance in Murine Hemophilia B Mice. Submitted to The 54^{th} ASH Annual Meeting, Atlanta, Ga. Dec. 8-12, 2012.

39. Kanaji, S., Fahs, S. A., Ware, J., Montgomery, R. R., and Shi, Q. Bleeding phenotype of murine Bernard Soulier Syndrome is potentially corrected by non-myeloablative hematopoietic stem cell transplantation. Submitted to The 54^{th} ASH Annual Meeting, Atlanta, Ga. Dec. 8-12, 2012.

1. A method of inducing immune tolerance against a protein of interest comprising the steps of:
(a) transducing hematopoietic stem cells with a gene for the protein of interest wherein the gene is operably connected to a platelet specific promoter, and
(b) transplanting the transfected cells of step (a) into a subject,
wherein the protein is expressed, and wherein the subject develops immune tolerance against the protein.

2. The method of claim 1 wherein the subject is immunologically sensitized to the protein prior to step (a).

3. The method of claim 1 wherein the subject is not immunologically sensitized to the protein prior to step (a).

4. The method of claim 1 wherein the transplanting of step (b) is a bone marrow transplant or an intravenous infusion of hematopoietic stem cells.

5. The method of claim 1 wherein the promoter of step (a) is a platelet specific promoter.

6. The method of claim 1 wherein the platelet specific promoter is the CD41 integrin alphaIIb promoter.

7. The method of claim 1 wherein the platelet promoter is selected from the group consisting of glycoprotein VI promoter, platelet factor 4 (PF4) promoter, glycoprotein Ib alpha promoter, glycoprotein Ib beta promoter, glycoprotein IX promoter and other platelet protein promoters.

8. The method of claim 1 wherein the protein is selected from the group consisting of clotting factor VIII, clotting factor IX, and von Willebrand factor.

9. The method of claim 1 wherein the protein is an alloantigen.

10. The method of claim 1 wherein the protein is a class I or class II human leukocyte antigen (HLA).

11. The method of claim 2 wherein the antigen against which the subject is sensitized is a human antigen known to be a target of autoantibodies that cause an autoimmune disease including but not limited to acquired hemophilia.

12. The method of claim 2 wherein the autoantigen against which the subject is sensitized is selected from the group consisting of, ADAMTS-13, nicotinic acetylcholine receptor, myelin basic protein, integrin alphaIIb/beta3 (GPIIb/IIIa) and glycoprotein Ib/V/IX.

13. The method of claim 2 wherein the acquired autoantibody is to factor VIII or factor IX.
